(19) United States
(12) Patent Application Publication
(10) Pub. No.: US 2006/0234370 A1
(43) Pub. Date: Oct. 19, 2006

(54) Analysis and culture apparatus

(75) Inventors: Juha Korpinen, Nokia (FI); Jouko Viitanen, Tampere (FI)

Correspondence Address: VENABLE LLP, P.O. BOX 34385, WASHINGTON, DC, 20045-9998, US

(73) Assignee: CHIP-MAN TECHNOLOGIES OY, TAMPERE (FI)

(21) Appl. No.: 10/553,997

(22) PCT Filed: Apr. 22, 2004
(86) PCT No.: PCT/FI04/50050

§ 371 Date: Oct. 21, 2005

(30) Foreign Application Priority Data
Apr. 22, 2003 (FI) 20030609

(51) Int. Cl. C12M 1/34

(52) U.S. Cl. 435/287.2

(57) ABSTRACT

An analysis and culture apparatus including several wells, which have a cover that closes the well to form a closed space, as well as at least one inlet passage and one outlet passage for introducing matter to a closed space and for removing it from a closed space, respectively. The cover is formed as a separate cap, which can be removably attached to the well, with which the well can be closed, and to which the inlet passage and outlet passage are integrated.

The invention relates to an analysis and culture apparatus comprising several wells, which have a cover that closes the well, as well as at least one inlet passage and one outlet passage for introducing matter into a closed space and for removing it from a closed space, respectively.

In microbiological cultures and different kinds of cell cultures it is known to use a closed culture environment, where the growing conditions are closely controlled. In this kind of apparatuses, there are typically several wells that comprise carefully selected culture medium.

A cell culture apparatus is presented in the U.S. Pat. No. 6,008,010. In the patent the cells are cultured in the wells of a well plate, but isolation from the surroundings in order to maintain carefully controlled conditions is implemented by means of a so-called incubator technique, where the well plate is placed in a closed chamber, whose top and bottom are transparent in order to make monitoring possible. The wells are open within the chamber, i.e. they exist in the same conditions determined by the chamber. The individual control of the culture environment and the input and outlet of materials is not possible for an individual well plate.

There is a need to feed medium from the outside the wells of this kind closed from the surroundings, and, at the same time, to remove excess medium. This kind of feed of a medium can be periodic or continuous, depending on the cell culture.

The German application publication 10019862 presents a method and an apparatus for changing a medium in cell cultures. In the apparatus, there is a perfusion cover that is transparent and smooth on its lower surface. The cover covers several wells of a well plate simultaneously. The cover is sealed at its sides against the outer periphery of the well plate, and on its lover surface there is an supply passage and an outlet passage at each well, which passages have as an extension a supply pipe and an outlet pipe that extend vertically down to the well, of which pipes the outlet pipe extends further down than the supply pipe. The supply passages are connected to the same supply connection; therefore, controlling conditions separately in the wells is not possible. In addition, the cover must always be dimensioned according to the size of the well plate. If one well is desired to be opened, the entire cover must be removed.

Publication U.S. Pat. No. 6,271,027 presents a cell and tissue culture apparatus, where there are culture wells in parallel rows, where the wells are connected in series in relation to the feed of a medium. A flexible ring placed inside the wells is typical for the structure of the wells. The ring is placed against the side wall of the well, and an inlet passage and an outlet passage are led through the ring. The well is closed from the top by a transparent cover placed within the ring. Both the ring and the cover are placed close to the bottom of the well in such a manner that a space closed from the surrounding remains between the bottom of the well and the cover.

The above-described structure is designed for cell culture purposes, and for that purpose the wells, flexible rings, as well as the cover have been manufactured, which must all be dimensioned correctly in relation to each other. The formation of a closed culture space from the above-mentioned elements requires a great deal of work in the assembly phase. In addition, connecting several wells in a series does not provide the possibility to control the conditions individually in one well.

The purpose of the invention is to present an analysis and culture apparatus which does not comprise the above-mentioned drawbacks. In order to attain this purpose, the apparatus is primarily characterized in that the cover is formed as a separate cap, which can be removably attached in the well, by means of which the well can be closed, and to which the inlet passage and the outlet passage are integrated.

When using a plug-type cap comprising both the inlet and outlet passage and a sealing structure, the well can be closed tightly from the surroundings by means of one piece to form a closed culture space without a difficult assembly phase. By dimensioning the diameter of the cap suitably, it can be used for existing microtitre plates. For example, standard plates of 12, 24, 48 or 96 wells can be formed into culture apparatuses comprising the corresponding number of closed culture spaces by means of the same number of identical caps, where the inlet and outlet passages are ready-made. To the outer surface of the cap to which the inlet and outlet passages are connected, it is possible to couple a supply pipe and an outlet pipe, respectively. It is possible to couple an individual supply pipe and outlet pipe to each cap, in which case especially the supply of the supply pipe can be completely independent from the supply of other caps and, correspondingly, from the supply of the supply pipes of the wells. Therefore, conditions in each well can be controlled individually, even in a 96-well plate, if necessary.

A structurally good solution is reached by forming the cap in such a manner that it comprises an insert part placed within the well, which is advantageously cylindrical. One or more inlet passages and one or more outlet passages can run in the depth direction through this insert part (in the depth direction of the well). The inlet and outlet passages can be formed by means of bores formed in the rigid material of the cap, in which case it is not necessary to use separate pipes or other channels in order to access the culture space inside the well. These bores can be connected to the connection apertures on the outer surface of the cap, to which the above-mentioned supply and outlet pipes will be connected.

The sealing is provided, in the simplest manner, by arranging a seal on the outer surface of the insert part, for example in the groove surrounding it, which seals the cap against the side wall of the well.

There is preferably a shoulder above the insert part, which comes against the upper surface of the plate and determines the inserting depth of the cap. This shoulder can be formed of the lower surface of a flange part forming the upper part of the cap. The connection apertures of the above-mentioned supply and outlet passage/passages can open on the outer surface of this flange part, for example on its periphery.

In order to optimize the monitoring of the culture space, there is a transparent window in the middle of the cap. This enables monitoring at least from above. If the bottom of the corresponding well is transparent, the well can be made transparent vertically to both directions, which enables, for example, illumination from the opposite direction of the monitoring direction, for example, illumination from below.

Other advantageous structure options are presented in the appended claims and in the following description.

In the following, the invention will be described in more detail with reference to the appended drawings, in which

FIG. 1 shows a side view of a cap,

FIG. 2 shows the cap seen from below,

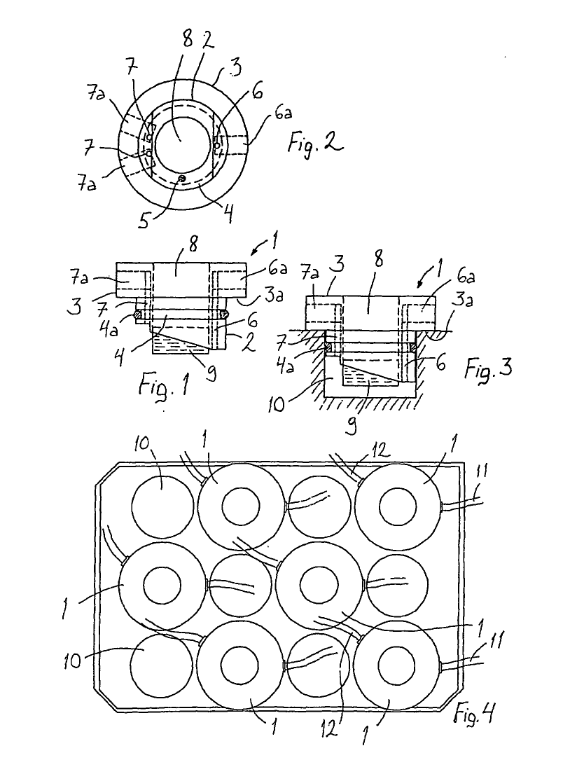

FIG. 3 shows the cap assembled in a corresponding well,

FIG. 4 shows the use of caps in an apparatus with several wells,

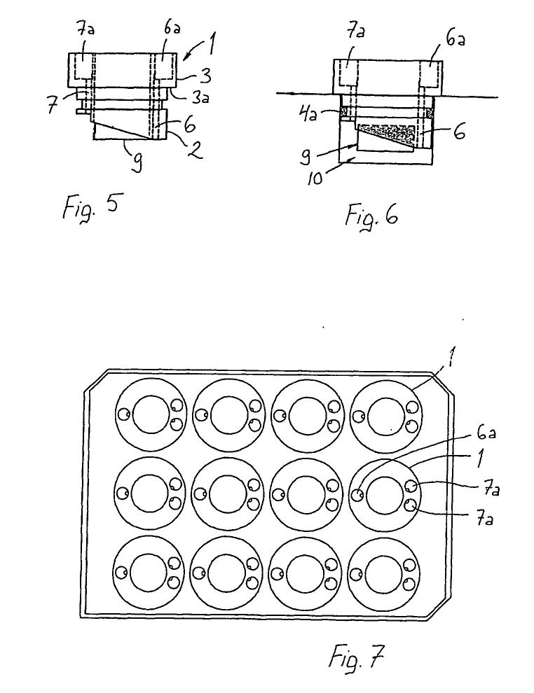

FIG. 5 shows a side view of a cap according to a second embodiment,

FIG. 6 shows the cap of FIG. 5 assembled in a corresponding well, and

FIG. 7 shows the use of caps of FIG. 5 in an apparatus with several wells.

FIG. 1 presents a side view of a cap, which is intended for an analysis and culture apparatus according to the invention. The analysis and culture apparatus here refers to an apparatus, where cells are cultured in controlled conditions by forming several closed culture spaces, to which it is possible to feed a medium having a determined composition according to a desired schedule, as well as to add substance required at different stages of the culture. The invention is not limited to the culture of only certain types of cells, but as an example can be mentioned, for example, the culture of different micro-organisms, as well as the maintenance and culture of cells of higher organisms. As an example of these can be mentioned the maintenance of gametes, for example, for the purposes of in vitro fertilization, or stem cell lines. A substantial part of the use of the apparatus is also the continuous monitoring by means of, for example, some imaging method; the images received during it can be stored, analyzed or used in controlling the living conditions within the closed culture space of the well. These methods are not described more in depth because they are not a part of the invention.

It is substantial to form a closed culture space in each well, to which there is a connection from the surroundings only through an inlet passage and through possible injection ports letting a medium pass only in injection situations. This is implemented by means of a cap 1 that can be introduce into a well (FIG. 1), and that comprises an insert part 2, whose cross-section perpendicular to the insertion direction corresponds to the horizontal section of the interior of the well. Since the wells of microtitre plates have normally round horizontal sections, the insert part 2 is cylindrical. A horizontal shoulder 3a circulates the insert part above it. The purpose of the shoulder is to lie against the upper edge of the well when the cap has been introduced into the well. The shoulder is formed of the lower surface of a wider flange part 3 situated above the insert part 2.

A little further down from the shoulder 3a, there is a groove 4 made in the side surface of the insert part 2 and circulating it. An O-ring seal 4a (presented in a side view) is placed in the groove.

At least one inlet passage 6 and outlet passage 7 run through the insert part in its longitudinal direction, which passages are formed as bores made in the material of the insert part. A bore here refers to a structure in the form of a channel closed in its cross-section, not necessarily made by machining, but also in connection with moulding. Both the inlet passage and the outlet passage are connected to a connection aperture 6a and 7a, respectively, opening in the flange part 3 in the radial direction, which apertures open on the cylindrical outer surface of the flange part 3.

As can be seen in FIG. 2, there are more than one outlet passage 7. Correspondingly, there can be more than one inlet passage 6. It is visible especially in FIG. 1 that the outlet passage 7 opens on the lower surface of the insert part 2 in vertical direction at a higher position than the inlet passage 6. Due to this, for example gases can escape more easily from the closed culture space, and they do not remain there to form bubbles.

In addition, it can be seen that the lower surface of the insert part 2 is inclined, starting from the horizontal lower surface that at a lower position, on which the inlet passage 6 opens, and ending at a vertical step, at the upper end of which there is a horizontal lower surface, on which the outlet passage 7 opens.

An aperture 8 runs centrally through the flange part 3 and the insert part 2 to the lower surface of the insert part from the upper surface of the flange part, which aperture is coaxial with the outer periphery of the insert part 2. A transparent window 9 is placed in the aperture 8, the lower surface of which window forms a part of the lower surface of the insert part. Thus, the window defines from above, together with the lower surface of the insert part 2 of a non-transparent material surrounding it, the culture space in a manner presented later. The window 9 is located in the lower end of the aperture 8. Thus, a visual connection is created through the aperture to the culture space, i.e. the cap is made transparent for the area extending from the upper surface of the cap to the lower surface of the insert part. The window is surrounded in a ring-like fashion by the rest of the lower surface of the insert part 2, which surface is directed upwards in a slanting manner, as described hereinabove, from the inlet passage towards the outlet passage.

FIG. 3 presents a cap 1 introduced in its place in one well 10 of a well plate. The well has a flat bottom and a side wall rising upwards from it and having a circular shape in the horizontal section. The internal volume of the well 10 is consequently cylindrical, and the insert part 2 of the cap 1 is placed in this volume at a depth which is defined by the shoulder 3a provided above the insert part 2. The shoulder is placed against the upper edge of the well, i.e. against the upper surface of the well plate. The cap 1 can be inserted from above into the well 10 in such a manner that the insert part 2 will be located within the well in a space defined by the side wall of the well, and the lower surface of the insert part 2 will lie at a certain distance from the bottom of the well 10 opposite to it.

The bottom of the well 10 is also transparent, in which case it is possible to look into the culture space defined between the bottom and the window 9, both from above and below the well, and this enables background illumination when the well is imaged by means of camera technique either continuously or periodically. The culture space created by the well and the cap can be used in such a manner that the lower surface 9 of the window determines the upper surface of the liquid in the culture space. Thus, there is free gas space outside the edges of the window 9 above the liquid surface. In this space, the gases are directed towards the outlet passage, thanks to the inclination of the part surrounding the window 9 in a ring-like manner.

The lower surface of the window 9 can also be somewhat inclined in such a manner that it rises towards the outlet passage 7. Thus, it can be ensured that no gas bubbles remain on the lower surface of the window 9.

In addition, it is possible to use a special injection passage 5, through which it is possible to bring a medium to the culture space independently of the supply passages. It can be formed of a bore running in the depth direction through the flange part 3 and the insert part 2, which bore is closed with an elastic material. In a normal situation, the material isolates the culture space from the environment, but allows the injection needle to permeate, i.e. it is a so-called injection rubber plug.

The cap can be manufactured of a plastic material by means of normal plastic machining methods. It is possible to manufacture the entire cap 1 of a transparent plastic, in which case the aperture 8 and the window 9 do not need to be made separately in it in order to make the cap transparent in the vertical direction.

FIG. 4 illustrates the use of caps in an apparatus with several wells. Each cap 1 and correspondingly each well 10 is allotted a supply pipe 11 of its own, which is connected to the corresponding inlet passage 6 in order to feed liquid matter to the culture space through the inlet passage 6. From each cap there is at least one outlet pipe 12, which is connected to the corresponding outlet passage 7 in the cap. It is possible to aspirate gas or liquid through the outlet passage by means of the outlet pipe. If the cap has connection apertures that are not needed, they can be plugged. The walls of the pipes are flexible to the extent that the pipe can be closed by pressing it flat. Thus, a simple press operating on the outside of the pipe can function as a valve without being in contact with the matter travelling in the pipe. The same effect can be reached by means of a so-called peristaltic pump, if it is arranged in contact with the pipe in such a manner that when the pump is stopped, at least one of its rolls presses the pipe shut. The outlet pipes can be closed in the same manner in order to isolate the culture space well from the surroundings. It can also be seen in FIG. 4 that a part of the wells 10 can be left empty. If there are two inlet passages in the caps, or one of the outlet passages has been selected as an inlet passage, it is possible to attach two inlet pipes to each cap 1 in order to feed different matters through their own pipes to the culture space.

An individual well 10 can be opened entirely by pulling the cap 1 out, without disturbing the operation of other wells. Similarly, if the purpose is to take only a part of an extensive well plate into use, only the selected wells need to be closed with caps 1, and the entire plate does not need to be covered (cover solution) or placed in a closed chamber (incubator solution).

FIGS. 5 and 6 present a cap in a view similar to FIGS. 1 and 3 (without a window in FIG. 5), the design of which cap is otherwise the same, but the inlet passage 6 and the outlet passage 7 run entirely in the axial direction (in the insertion direction of the insert part) through the cap in such a manner that the connection apertures 6a and 7a form axial extensions of the bores and open on the upper surface of the flange part 3 instead of the peripheral surface. In view of the manufacturing technique, this is easier to implement, especially if the cap is manufacture by means of compression moulding technique. It is obvious that the cap of FIG. 5 can also be manufactured entirely of the same transparent plastic material, including the window, while the design remains the same. Similarly, it is possible to form an injection passage of one passage by plugging it with an injection rubber plug, because there is a straight line from the outer surface of the cap to the culture space, through which a needle can be brought in. FIG. 7 presents the placement of the caps 1 according to FIGS. 5 and 6 in the wells 10 of the well plate as a top view.

As was mentioned earlier, the apparatus can be used for many culture methods of living cells. The supply of matters to the culture spaces and their removal from there can be continuous or periodic, depending on the type of the cultured cells and/or their phase of living. Instead of purely culture or maintenance purposes, the apparatus can also be used for analyzing, for example in order to examine how foreign substances (drugs, toxics, etc.) affect the cells, such as different cell cultures and micro-organisms. Also, there can be an optional number of inlet passages and outlet passages, for example, there can also be two or more inlet passages. Similarly, at least one of the two or more outlet passages can be used, if necessary, as an inlet passage as well, and at least one of the two or more inlet passages can be used as an outlet passage as well, if necessary, by changing the connections to the pipes. This increases adaptability and alternatives in the removal and supply of different matters.

1-15. (canceled)

16. An analysis and culture apparatus comprising several wells having depth directions, the wells having separate caps to close the wells to form closed spaces, wherein the caps can be removably attached in the wells and have an inlet passage and an outlet passage integrated in the cap for introducing matter to the closed space and for removing it from the closed space, respectively.

17. The analysis and culture apparatus according to claim 16, wherein the cap comprises an insert part that can be placed in the well, said insert part having a lower surface, and a shoulder supported by an upper edge of the well.

18. The analysis and culture apparatus according to claim 17, wherein the cap comprises a flange part being wider than the insert part and having a lower surface forming said shoulder.

19. The analysis and culture apparatus according to claim 17, wherein there is a seal around the insert part, which seal is placed against a side wall of the well.

20. The analysis and culture apparatus according to claim 18, wherein there is a seal around the insert part, which seal is placed against a side wall of the well.

21. The analysis and culture apparatus according to claim 17, wherein the inlet passage and the outlet passage open on the lower surface of the insert part.

22. The analysis and culture apparatus according to claim 19, wherein the inlet passage and the outlet passage open on the lower surface of the insert part.

23. The analysis and culture apparatus according to claim 21, wherein the inlet passage opens at a lower position than the outlet passage.

24. The analysis and culture apparatus according to claim 18, wherein, in order to couple the inlet and outlet channels to the cap, there are connection apertures on the outer surface of the flange part.

25. The analysis and culture apparatus according to claim 17, wherein there is an inclined portion on the lower surface of the insert part.

26. The analysis and culture apparatus according to claim 21, wherein there is an inclined portion on the lower surface of the insert part.

27. The analysis and culture apparatus according to claim 16, wherein the cap is transparent in the depth direction of the well.

28. The analysis and culture apparatus according to claim 17, wherein the cap is transparent in the depth direction of the well.

29. The analysis and culture apparatus according to claim 16, wherein separate input channels are brought to the caps placed in the wells.

30. The analysis and culture apparatus according to claim 17, wherein separate input channels are brought to the caps placed in the wells.

31. A cover for an analysis and culture apparatus comprising several wells, said cover being a separate cap, which can be removably attached to an indivicual well in order to close it, and to which an inlet passage and an outlet passage for bringing matter to the well and for removing it from the well, respectively, are integrated.

32. The cover according to claim 31, wherein the cap comprises an insert part, which is intended to be placed in the well, said insert part having a lower surface, and a shoulder above the insert part, which is intended to be supported by an upper edge of the well.

33. The cover according to claim 32, wherein the cap comprises a flange part being wider than the insert part and having a lower surface forming said shoulder.

34. The cover according to claim 32, wherein there is a seal around the insert part, which is intended to be placed against a side wall of the well.

35. The cover according to claim 33, wherein there is a seal around the insert part, which is intended to be placed against a side wall of the well.

36. The cover according to claim 32, wherein the inlet passage and the outlet passage open on the lower surface of the insert part in the cap.

37. The cover according to claim 33, wherein the inlet passage and the outlet passage open on the lower surface of the insert part in the cap.

38. The cover according to claim 34, wherein the inlet passage and the outlet passage open on the lower surface of the insert part in the cap.
